(19) United States
(12) Patent Application Publication
(10) Pub. No.: US 2025/0041238 A1
(43) Pub. Date: Feb. 6, 2025

(54) METHOD FOR LAGRE-SCALE PRODUCTION OF LARGE-SIZE LNPs

(71) Applicant: eTheRNA immunotherapies NV, Niel (BE)

(72) Inventors: George R. DAKWAR, Niel (BE); Phillip CHALLIS, Niel (BE)

(73) Assignee: eTheRNA immunotherapies NV, Niel (BE)

(21) Appl. No.: 18/719,001

(22) PCT Filed: Dec. 12, 2022
(86) PCT No.: PCT/EP2022/087505

§ 371 Date: Jun. 12, 2024

(30) Foreign Application Priority Data
Dec. 23, 2021 (EP) 21217376.9

(51) Int. Cl. A61K 9/51

(52) U.S. Cl. CPC A61K 9/5192; A61K 9/5123

(57) ABSTRACT

The present invention relates to the field of lipid nanoparticles (LNP); more specifically a method for the large-scale production of large-sized LNPs. The method of the present invention produces LNPs which are characterized in having a minimal average diameter of about 140 nm, via a scalable method to produce large volumes of LNP's quickly. The present invention provides a scalable, large scale. GMP-compliant method for the production of large LNPs for the delivery of active agents, more in particular for the immunogenic delivery of nucleic acid molecules, specifically mRNA.

### FIELD OF THE INVENTION

The present invention relates to the field of lipid nanoparticles (LNP); more specifically a method for the large-scale production of large-sized LNPs. The method of the present invention produces LNPs which are characterized in having a minimal average diameter of about 140 nm, via a scalable method to produce large volumes of LNP's quickly. The present invention provides a scalable, large scale, GMP-compliant method for the production of large LNPs for the delivery of active agents, more in particular for the immunogenic delivery of nucleic acid molecules, specifically mRNA.

### BACKGROUND TO THE INVENTION

One of the major challenges in the field of targeted delivery of biologically active substances is often their instability and low cell penetrating potential. This is specifically the case for the delivery of nucleic acid molecules, in particular (m) RNA molecules. Therefore, proper packaging of active agents, such as nucleic acid molecules is crucial for adequate protection and delivery. Hence, there is a continuous need for methods and compositions for packaging biologically active substances, such as nucleic acids. In that respect, lipid-based nanoparticle compositions such as lipoplexes and liposomes have been used as packaging vehicles for biologically active substances to allow transport into cell and/or intracellular compartments.

Another type of packaging compositions are lipid nanoparticles (LNPs). LNPs are typically composed of a mixture of 4 lipids—a cationic or ionizable lipid, a phospholipid, a sterol and a PEGylated lipid—and have been developed for the non-immunogenic delivery of siRNA and mRNA to the liver after systemic administration. To evoke optimal hepatocyte uptake and expression, these LNPs typically display small sizes between 70-100 nm (Li et al., Nanoletters 2015; Thess et al. Mol the 2015; Kauffman et al. Biomaterials 2016).

LNPs have also been used for the immunogenic delivery of antigen encoding mRNA in muscle or dermis (Richner et al. Cell 2017; Liang et al., Mol Ther 2017). In this case, again small size (80-120 nm) LNPs are typically used since such small sizes have been shown to be crucial for immunogenicity. Moreover, small sized LNPs may efficiently reach the injection draining lymph node, whereas larger sized LNP's are more frequently retained at the injection site (see Reichmuth et al., 2016).

It has recently been found that, in contrast to the general belief, although small-sized particles are beneficial for LNP mediated delivery, nanoparticles having a diameter of more than 140 nm, correlate with an enhanced delivery of active agents (such as mRNA) to the spleen, in particular after intravenous administration. Thus, there is an interest in a method for the production of large LNPs for this purpose.

In the prior art, methods have been described for the production of these large (>100 nm) LNPs, although they are identified for their negative trait of being less potent and encapsulating nucleic acid as successfully as their smaller counterparts (MacLachlan, I., 2007 and Chen, Sam, et al. 2016). Also; large-scale production of large-sized GMP-compliant LNPs is still a challenge. There are no methods to consistently produce large batches of potent large-sized LNP with a high encapsulation rate in the prior art. Since these large LNPs are particularly beneficial for delivery of active agents, such as nucleic acids to the spleen, a method to consistently produce LNPs with an average diameter of at least 140 nm on a pharmaceutically relevant scale would be beneficial.

### SUMMARY OF THE INVENTION

In a first aspect the present invention provides a method for the large-scale production of a composition comprising LNPs according to the invention, comprising:
a) mixing a first composition comprising an ionisable lipid, a phospholipid, a sterol, and a PEG lipid in an organic solvent with a second composition comprising one or more active agents (such as nucleic acids) in an acidic buffer, thereby obtaining a post-mixing bulk,
b) applying tangential flow filtration (TFF) to said post-mixing bulk of steps a),
c) applying a filtration step in an aqueous solution having pH<6.5 to said composition of step b),
d) adding a cryobuffer comprising a cryoprotectant to said composition of step c),
e) applying a filtration step in an aqueous solution having pH>6.5 to said composition of step d),
f) sterile filtration of said composition of step e), thereby obtaining a composition of lipid nanoparticles having an average diameter of at least 140 nm and comprising said active agent,
characterised in that said TFF of step b) is performed at a shear rate of less than 4000/s, and wherein the LNPs comprise less than 1 mol % of PEGylated lipid.

In a specific embodiment of step a), the mixing flow rate of said first composition is lower than the mixing flow rate of said second composition.

In another specific embodiment of step a), the ratio of the mixing flow rates between the first and the second composition is about 1:2.

In another specific embodiment, said tangential flow filtration of step b) comprises a first ultrafiltration step which is performed at a lower shear rate compared to the subsequent tangential flow filtration step(s).

In another specific embodiment, said tangential flow filtration of step b) comprises a first ultrafiltration step being performed at a shear rate of <3000/s whilst the subsequent tangential flow filtration steps are performed at a shear rate of <4000/s.

In another specific embodiment, said organic solvent is chloroform or an alcohol such as methanol or ethanol; in particular ethanol.

In another specific embodiment, said cryoprotectant is selected from the list comprising: maltose, maltodextrin, dextran, mannitol, glucose, trehalose or sucrose; in particular trehalose or sucrose.

In yet a further embodiment, said active agent is selected from the list comprising small molecules and biomolecules, in particular antibodies, peptides, proteins and/or nucleic acids; more in particular mRNA or DNA molecules.

In another specific embodiment, said cryobuffer comprises a cryoprotectant and one or more of TRIS, citrate, or PBS; in particular TRIS and/or PBS.

In another specific embodiment, said first composition of step a) is at a temperature of 30-45° C.

In another specific embodiment, said composition of a) is diluted before step b) and/or said composition of step e) is diluted before step f).

In another specific embodiment, the filter size/cutoff of said filtration in steps c and/or e) is 0.20 μm.

In yet a further embodiment, said aqueous solution of steps c) and/or e) is water, in particular water for injection.

In another specific embodiment, said filtration of steps c) and/or e) is a clarifying filtration.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1 Size distribution derived from transmission electron microscopy analysis of the samples of table 1 of example 1. The average particle size is in the 150-200 nm in diameter range.

FIG. 2 Size distribution derived from transmission electron microscopy analysis of the samples of table 2 of example 2. The average particle size is in the 150-200 nm in diameter range.

FIG. 3: Size distribution after TFF in water (average size: 141 nm in diameter, PDI: 0.49)

FIG. 4: Size distribution after filtration in water (average size: 92.5 nm, PDI: 0.45)

FIG. 5: Size distribution after sterile filtration-LNP in cryobuffer (average size: 142 nm, PDI: 0.05)

### DETAILED DESCRIPTION OF THE INVENTION

As already detailed herein above, the present invention provides a scalable, GMP-compliant method to produce lipid nanoparticles (LNPs) having a diameter which is larger than generally used in the field. These LNPs have been found to be highly suitable for the delivery of active agents, more in particular for the immunogenic delivery of nucleic acids, specifically mRNA; for which a correlation was found with enhanced delivery to the spleen following administration (e.g. intravenous administration) to a subject in need thereof. “Immunogenic delivery of nucleic acid molecules” means delivery of nucleic acid molecules to cells whereby contact with cells, internalization and/or expression inside the cells of said nucleic acids molecules result in induction of an immune response. This large-scale production is achieved in accordance with the method as identified herein and in particular uses a surprisingly low shear rate tangential flow filtration (TFF) and a surprisingly low percentage of PEGylated lipid in the method.

Therefore, in a first aspect, the present invention provides a method for large scale production of LNPs comprising an ionisable lipid, a phospholipid, a sterol, a PEG lipid and one or more active agents wherein said LNPs have an average diameter of about 140 nm or more.

In particular, the invention provides methods for the large-scale production of GMP compliant large-size LNPs, comprising:
a) mixing a first composition comprising an ionisable lipid, a phospholipid, a sterol, and a PEG lipid in an organic solvent with a second composition comprising one or more active agents in an acidic buffer, thereby obtaining a post-mixing bulk,
b) applying tangential flow filtration (TFF) to said post-mixing bulk of steps a),
c) applying a filtration step in an aqueous solution at pH<6.5 to said composition of step b), d) adding a cryobuffer comprising a cryoprotectant to said composition of step c),
e) applying a filtration step to the composition of step d) in an aqueous solution at a pH>6.5
f) sterile filtration of said composition of step e), thereby obtaining a composition of lipid nanoparticles comprising an active agent; and having an average diameter of at least 140 nm.
characterised in that the TFF of step b) is performed at a shear rate of less than 4000/s, and wherein the first composition of step a) contains low percentage (<1%) of PEGylated lipid.

A lipid nanoparticle (LNP) is generally known as a nanosized particle composed of a combination of different lipids. While many different types of lipids may be included in such LNP, the LNPs produced by the method of the present invention are typically composed of a combination of an ionisable lipid, a phospholipid, a sterol and a PEG lipid.

As used herein, the term “nanoparticle” refers to any particle having a diameter making the particle suitable for systemic, in particular intravenous, administration of active agents in particular, nucleic acids, typically having a diameter of less than 1000 nanometers (nm).

In some embodiments, the LNPs of the invention have an average diameter of less than 600 nm. In some embodiments, the LNPs of the invention have an average diameter of less than 400 nm, but in any case the nanoparticles of the present invention have an average diameter of more than about 140 nm. In a specific embodiment, the LNP's of the invention have an average diameter of about 140 nm or more, about 150 nm or more, about 160 nm or more, about 170 nm or more, about 180 nm or more, about 190 nm or more; or about 200 nm or more. For the sake of clarity, where a mixture of multiple LNP's is used, the average diameter as referred to, is meant to be the average diameter of said multiple LNP's. In said instance, for example the mixture may contain some LNP's having a smaller average diameter than 140 nm, in as far as the remainder of the LNP's has an average diameter larger than 140 nm to result in an average diameter of at least 140 nm of all LNP's together. Whenever used in this application the term “diameter” is meant to be the “average diameter”, even if it is not specifically specified. Whenever used in this application the term “large-sized LNPs” refers to LNPs having a diameter which is greater than about 140 nm.

Particle size and polydispersity index of LNPs can be measured using the Malvern Zetasizer dynamic light scattering (DLS) equipment (Malvern Panalytical Ltd., UK). The particle size values presented in this application represent the Z-Average diameter of the particle size distribution. Since DLS is a diffusion-based technique, where a distribution of diffusion coefficients is converted to particle size distribution, the viscosity of the media in which the LNP-mRNA plays an important role to obtain the correct average diameter.

Samples are let to equilibrate for 120 seconds at 23° C., and measurements are performed based on the LNP-mRNA as follows: before addition of cryobuffer, the LNP-mRNA samples are in water and the viscosity value of water at 23° C. is used to measure particle size. After addition of cryobuffer, the measured viscosity value which takes into consideration the presence of cryoprotectant at 23° C. is used. Accordingly, in a particular embodiment the LNPs of the present invention have an average diameter of at least 140 nm as measured using dynamic light scattering (DLS) considering the viscosity value of the buffer in which the LNPs resides at 23° C. Alternatively, size distribution of the LNPs may be determined using other suitable techniques, such as by Transmission Electron Microscopy (TEM), as detailed in example 2.

The method of the present invention comprises a number of steps to produce LNPs having an average diameter of more than about 140 nm. The method is scalable and can be used to produce industrially significant numbers of said LNPs.

In the context of the present invention, the term “large-scale production” is meant to be production on an industrial scale, in particular the production of large batches of LNPs, such as containing a total load of at least 250 mg of active agent (e.g. mRNA); in particular at least 500 mg, such as about 1 g, about 2 g or even more,

In the context of the present invention the term “ionisable” (or alternatively cationic) in the context of a compound or lipid means the presence of any uncharged group in said compound or lipid which is capable of dissociating by yielding an ion (usually an H+ ion) and thus itself becoming positively charged. Alternatively, any uncharged group in said compound or lipid may yield an electron and thus becoming negatively charged. In many cases, the positive charge is a result of the presence of a quaternary nitrogen atom.

In the context of the present invention any type of ionizable lipid can suitably be used.

In the context of the present invention the term “phospholipid” is meant to be lipid molecule consisting of two hydrophobic fatty acid “tails” and a hydrophilic “head” consisting of a phosphate groups. The two components are most often joined together by a glycerol molecule, hence, the phospholipid of the present invention is preferably a glycerol-phospholipid. Furthermore, the phosphate group is often modified with simple organic molecules such as choline (i.e. rendering a phosphocholine) or ethanolamine (i.e. rendering a phosphoethanolamine).

Suitable phospholipids within the context of the invention can be selected from the list comprising: 1,2-Dioleoyl-sn-glycero-3-phosphoethanolamine (DOPE), 1,2-Dioleoyl-sn-glycero-3-phosphocholine (DOPC), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 5 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phosphorac-(1-glycerol) sodium salt (DOPG), sphingomyelin, and mixtures thereof.

In the context of the present invention, any suitable sterol may be used, such as selected from the list comprising cholesterol, ergosterol, campesterol, oxysterol, antrosterol, desmosterol, nicasterol, sitosterol and stigmasterol; preferably cholesterol.

In the context of the present invention, the term “PEG lipid” or alternatively “PEGylated lipid” is meant to be any suitable lipid modified with a PEG (polyethylene glycol) group. In a particular embodiment, said PEG lipid is selected from the list comprising: PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. More specific examples of such PEG lipids encompass C14-PEG2000 (1,2-Dimyristoyl-rac-glycerol, methoxypolyethylene glycol-2000 (DMG-PEG2000)) and C18-PEG5000 (1,2-Distearoyl-rac-glycerol, methoxypolyethylene glycol-5000 (DSG-PEG5000)).

As used herein, apart from the various lipids as defined herein, the first composition further comprises an organic solvent. Such organic solvent is required to keep the lipids encompassed therein in solution. Organic solvents which may be suitably used within the context of the invention, are for example chloroform or alcohols, such as methanol or ethanol; in particular ethanol.

Moreover, in a further specific embodiment, said first composition is preferably kept at a slight elevated temperature, meaning a temperature being above room temperature but below boiling point. Such elevated temperature may for example be between 30° C. and 60° C., such as between 30° C. and 50° C., more in particular between 30° C. and 45° C. This slightly elevated temperature improves the solubility of the lipids dissolved therein, thereby increasing the efficiency of the mixing process, which was found to be advantageous in the LNP production process.

In the context of the present invention, the term tangential flow filtration (TFF) is meant to be a method for separation and purification of biomolecules which is well known in the art. A TFF system typically comprises a filter device such as a capsule, cassette and holder, or hollow fiber module, a pump, tubing, a valve or clamp, and a fluid reservoir, and optionally a pressure gauge, Such a TFF system can be used continuously even with high concentrations of active agents (e.g. nucleic acids) without clogging of the filter membrane pores. The use of a TFF system is advantageous as it allows high-throughput purification of the LNPs to be purified.

TFF in particular refers to the diffusion of a liquid through said filter device, wherein the feed stream flows parallel to the membrane face. Pressure is applied to a feed reservoir so that a stream passes along the filter, with larger particles not being permitted through the filter. To prevent these particles blocking the filter, they are recycled back into said feed reservoir. TFF is carried out at a particular shear rate, which is the rate at which the feed is applied to the filter. The key applications of TFF in LNP manufacturing are concentration and diafiltration (buffer exchange and desalting).

Filter membranes suitable for use in LNP purification can be of any type of materials suitable for said purpose and thus, not interacting with the LNPs be purified. Examples for materials of filter membranes include modified or unmodified polyethersulfone (mPES or PES, polyvinylidene fluoride (PVDF), cellulose acetate, nitrocellulose, mixed cellulose ester (ME), ultra-high MW polyethylene (UPE), polyfluorotetraethylene (PTFE), nylon, polysulfone (PS), polyacrilonitrile, polypropylene, polyvinyl chloride, polyvinylidene difluoride (PVDF), and combinations thereof. Filter membranes are characterized by their molecular weight cut-off (MWCO) value, which refers to the lowest molecular weight of particles in daltons of which 90% are retained by the filter. Preferably, a filter membrane has a pore size that is appropriate for retaining LNPs while allowing components of smaller size than the MWCO and thus, the pore size, to pass through the filter membrane as permeate.

In some embodiments of the present invention, a filter membrane with a molecular weight cut-off between 100 and 500 kDa is used for TFF, preferably of between 100 and 300 kDa, more preferably of between 100 and 200 kDa, most preferably of about 100 kDa. The filter membrane used in TFF is important as the pore size of the filter membrane determines the size of particles such as LNPs and components that can pass the filter membrane and are thus comprised in the permeate.

Processes using TFF can furthermore be characterized in view of different variables with the two most important ones being the transmembrane pressure, the flow/shear rate.

The “transmembrane pressure” (TMP) refers to the driving force that drives components through the filter membrane. In some embodiments, the transmembrane pressure is in the TFF step is between 100 mbar and 700 mbar, preferably between 100 mbar and 500 mbar, more preferably between 200 mbar and 400 mbar, alternatively the pressure may be expressed as psi, such as ranging from about 2 psi to about 8 psi.

The term “flow” refers to the volume of solution flowing through a TFF system and especially in the filter membrane area with a “flow rate” or “shear rate” referring to the solution volume flowing through said system during a given time. The term is meant to be the rate required in order to re-circulate the mRNA molecules through the TFF system and to sweep away the LNPs from the filter surface Thus the flow/shear rate or crossflow velocity refers to the rate of a solution flow across the filter membrane. In some embodiments, the shear rate in the TFF steps of the invention is typically less than 4000/s. In a particular embodiment, the first concentration step is performed at a shear rate of about 2800/s, corresponding to about 1300 mL/min, whereas the diafiltration and second ultrafiltration is performed at a shear rate of about 3800/s, corresponding to about 1400 ml/min.

Prior art methods for LNP production use shear rates higher than those as outlined in the method of the current invention.

The shear rate is a measure of the speed at which a stream is applied to a the TFF fliters, and is typically above 5000/s in the prior art, but is <4000/s in the method of the present invention.

In particularly preferred embodiments of the present invention, the method for purifying LNPs is performed using continuous TFF. Continuous TFF refers to a TFF system in which the retentate is used as feed for another round of TFF. Herein, the term “feed” refers to a solution or suspension comprising the LNPs to be purified. Hence, the initial feed comprising the LNPs and is subjected to a first round of filtration using TFF, and the obtained retentate comprising the LNPs is used again as feed for another round of TFF by circulating at least the LNPs. This is advantageous for enhancing the level of purification due to repeated steps of purification in an automated way while reducing the risk of losing LNPs due to a transfer between systems.

Thus a tangential flow filtration system as used in the invention may comprises multiple filtration steps, which may each be performed at different flow rates. It was particularly found that a first ultrafiltration step performed at a lower flow rate than the subsequent filtration steps is advantageous in the context of the invention. Accordingly, the present invention provides a method in which the tangential flow filtration in step b) comprises a first ultrafiltration step being performed at a shear rate of <3000/s whilst the other tangential flow filtration steps are preferably performed at a shear rate of <4000/s. In a very specific embodiment, the first ultrafiltration step is performed at a shear rate of about 2800/s and the remainder of the other ultrafiltration steps are performed at a shear rate of about 3800/s.

Shear rates as used in the context of the invention are lower compared to those typically used in the prior art. It was found that these lower shear rates are advantageous in the process for obtaining large-sized particles.

In the context of the present invention, the term “clarifying filtration step” is commonly used in GMP processes. This filter step has the purpose of collecting a liquid composition that is as free from solid impurities as possible. The filters used herein are specifically designed in such a way as to remove the very smallest particles suspended in the liquid. Multiple systems have been developed to achieve this objective. For example, filters may be designed using wires or fibres that are spaced very closely together.

In particular the first filtration step as applied in the present invention in acidic watery conditions is meant to provide a purification in which particles that have not correctly formed during the TFF step are removed, such as those particles which are too large for further processing. The second step of filtration in the cryobuffer is also termed the ‘clarifiying filtration step’ and is meant to reduce the so-called bio-burden before storage.

In the context of the present invention, the term “sterile filtration” is also a step which is a filtration method commonly used in GMP processes. This filter step has the purpose of removing particulate and contamination from fluids. Typically, filtering through a 0.2 micrometer pore size is considered necessary to generate a sterile filtrate.

In the context of the present invention, the term “cryobuffer” is meant to be a solution, which will not solidify on cooling and which does not substantially harm the integrity and/or stability of the molecules contained therein. In particular, the cryobuffer of the present invention comprises a cryoprotectant and one or more of citrate, Tris and PBS; in particular Tris and/or PBS.

In the context of the present invention, the term “cryoprotectant” is meant to be a substance used to protect biological tissue from freezing damage. In a specific embodiment of the present invention the cryoprotectant may be selected from the list comprising sugars such as maltose, maltodextrin, dextran, mannitol, trehalose, glucose or sucrose; in particular trehalose or sucrose.

In the context of the present invention, the term “mixing flow rate” is meant to be the rate at which the compositions are added to the mixing compartment. In an advantageous embodiment of the present invention the mixing flow rate of the first and second composition is different from one another. More in particular, the mixing flow rate of the first composition is preferably lower compared to that of the second composition. In a very specific embodiment, the ratio of mixing flow rates of the first composition compared to that of the second composition is about 1:2, meaning that the flow rate of the second composition is about double that of the first composition. Alternatively, the ratio of mixing flow rates of the first compared to the second composition is about 1:3, 1:4, 1:5; preferably about 1:2.

The aim of mixing is to achieve thorough and rapid mixing of multiple samples (i.e. lipid phase and nucleic acid phase) in a device. Such sample mixing is typically achieved by enhancing the diffusion effect between the different species flows. Thereto several mixing devices can be used, such as for example reviewed in Evers et al., 2018.

The inventors have found that the method of the present invention is particularly suitable for the large-scale production of LNPs with a diameter greater than about 140 nm. The method of the present invention is GMP compliant. The method of the present invention is scalable. Methods featured in prior art can only develop large LNPs on a small, laboratory scale and are not scalable. Hence the present invention provides a method for the production of large LNPs at an industrially significant volume, wherein the LNPs are GMP-compliant.

An “active agent” as used in the context of the present invention refers to any component which may suitably be formulated in an LNP, such as small molecules or biomolecules. The invention is particularly suitable for the formulation of biomolecules such as antibodies, proteins, peptides, nucleic acids, . . . .

A “nucleic acid” in the context of the invention is a deoxyribonucleic acid (DNA) or preferably a ribonucleic acid (RNA), more preferably mRNA. Nucleic acids include according to the compositions of the method of the invention genomic DNA, cDNA, mRNA, recombinantly produced and chemically synthesized molecules. The invention may also suitable be used in the formulation of siRNA molecules, circular RNA/DNA, self-amplifying RNA, . . . .

A nucleic acid may, according to method the invention be in the form of a molecule which is single stranded or double stranded and linear or closed covalently to form a circle. A nucleic can be employed for introduction into, i.e. transfection of, cells, for example, in the form of RNA which can be prepared by in vitro transcription from a DNA template. The RNA can moreover be modified before application by stabilizing sequences, capping, and/or polyadenylation.

To avoid any misunderstanding the LNPs produced via the method of the present invention may comprise a single active agent, or they may comprise multiple active agenst, such as a combination of one or more active agents, e.g. nucleic acid molecules encoding immune modulating proteins and/or one or more nucleic acid molecules encoding antigen- and/or disease-specific proteins.

As detailed herein above, the method of the present invention comprises multiple filtration steps which were found to contribute significantly in the process of obtaining large-sized nanoparticles. These steps may selectively enrich for such large-size particles, without compromising the stability and integrity thereof. This is a process which has previously not been considered feasible for the large-scale production of LNPs. During such process, it may be advantageous to apply a dilution step in a suitable buffer/solution prior to or after one or more of the filtration steps used herein. Accordingly, in a further embodiment, the composition of a) may be diluted before step b). In a further embodiment, the composition of e) may be diluted. The applied dilution factor may be dependent on the concentration of the solution to be filtered and can be determined by a skilled artisan.

For example, for the first dilution of the post-mixing bulk, it was found that a 6× dilution in water provides a stable post-mixing bulk in terms of particle size and encapsulation efficiency as a function of time. In particular, the post-mixing bulk in the organic solvent and acidic buffer is slightly unstable and tends to aggregate. Therefore, for better storage/stability between different steps of the method, such as transfer to a cleanroom and/or handling large volumes, the dilution step ensures that the bulk is stable for a longer period of time until the TFF starts

For the second dilution it may be desirable to dilute to the desired concentration of the product in the vial, for example to a concentration typically used in the field, such as 100 μg/ml

Using the larger LNPs as produced by the method of the present, a pharmaceutical composition or a vaccine for the immunogenic delivery of said one or more active agents (e.g. nucleic acids molecules) can be achieved. This is due to their preferential targeting to the spleen. However, methods of the prior arts to obtain large LNPs are small scale, laboratory methods.

The surprising aspects of the method of the invention which leads to large-scale production of large LNPs is the multiple filtration steps in combination with the low shear rate used in the TFF stage, along with the low percentage (<1%) of PEGylated lipid in said first composition, as detailed in the examples part.

### EXAMPLES

### Example 1—Production Method for Large-Sized LNPs

In this example the method of the present invention was carried out to investigate the possibility of developing large LNPs.

A mixture of Coatsome SS/EC (ionizable lipid), DOPE (phospholipid), cholesterol and a DSG-PEG 2000 lipid (PEGylated lipid) were mixed in an ethanol buffer to pre-defined molar ratios of 64:8:27:0.5, to a total lipid concentration of 10.13 mg/mL. This mixture was then heated to 41° C. in a water bath.

A watery solution containing mRNA was prepared by diluting a stock of nucleic acids with 200 mM Sodium Acetate to obtain a solution having an mRNA concentration of 0.126 mg/ml mRNA in 100 mM sodium acetate buffer at a pH of 4.0.

The lipid and mRNA mixtures from the above steps were mixed in a mixing device including pumps. The rates at which the lipids and mRNA composition were pumped into the mixing device was 66.7 mL/min and: 133.3 mL/min respectively. This total mixture was then diluted six times in water for injection, before being subjected to a buffer exchange and concentration via tangential flow filtration (TFF). This TFF consisted of three substeps. Firstly, concentration (70×) which is achieved using a hollow fibre cartridge with a 0.5 mm internal diameter and a 100 Kda cutoff at a shear rate of 2800/s. Secondly, buffer exchange at a shear rate of 3800/s against 4 wash volumes of water for injection. The final stage is concentration (2×) which is achieved using a hollow fibre cartridge with a 0.5 mm internal diameter and a 100 Kda cutoff at a shear rate of 3800/s.

At this stage the mixture underwent a clarifying filtration in water for injection through a Sartopore 2 (0.45+0.2 μm) sterile filters, before being diluted in a 466 mM Trehalose Dihydrate, 18 mM Tris (Hydroxymethyl)aminomethane (Trometamol) buffer, containing a 137 mM Sodium Chloride, to a pH of 7.4. At this point a drug product has been developed.

This drug product underwent another clarifying filtration in water for injection through a Sartopore 2 (0.45+0.2 μm) sterile filters before being diluted in 466 mM Trehalose Dihydrate, 18 mM Tris (Hydroxymethyl)aminomethane (Trometamol) containing 137 mM Sodium Chloride at a pH 7.4 to a concentration of 0.110 mg/ml mRNA. A final sterilisation filtration through a Sartopore 2 (0.45+0.2 μm) sterile filter was carried out.

The method as outlined above was repeated 2 further times. Particle Size, mRNA content and encapsulation, lipid content ratio, pH and osmolality were measured at different stages throughout the method. For the 3 repeats the analysis can be seen in Table 1 (250 mg total mRNA batch), Table 2 (250 mg total mRNA batch) and Table 3. (100 mg total mRNA batch).

TABLE 1

| | Particle Size Analysis | | mRNA analysis | |
|---|---|---|---|---|
| | Large-scale batch 1 | | Large scale batch 1 | |
| | Z- | | mRNA | |
| | Ave | | content | Encapsulation |
| Sample | (nm) | Pdl | (μg/ml) | (%) |
| Post-Mixing | 52.9 | 0.17 | | |
| Post dilution with water (6x) | 50.8 | 0.17 | | |
| Post first ultrafiltration | 52.2 | 0.31 | | |
| Post diafiltration | 73.8* | 0.292* | | |
| Post second ultrafiltration | 82.4 | 0.27 | 2122 | 93.5 |
| Post CF in water | 80 | 0.24 | 2404 | 95.9 |
| Post dilution with cryobuffer | 139.1 | 0.11 | | |
| Post CF in cryobuffer | 136.1 | 0.10 | 202 | 95.6 |
| Post dilution to label claim | 143.9 | 0.05 | 113 | 95.2 |
| Post dilution to label claim (overnight | 169.1 | 0.02 | 115 | 95.2 |
| storage at 2-8° C.) | | | | |
| Post SF | 167.8 | 0.02 | 112 | 96.1 |
| Day 1 filling / Day 1 filling overnight | 156.8 | 0.04 | 114 | 93.8 |
| storage at 2-8° C. | | | | |
| Day 2 filling / Day 2 filling overnight | 156.8 | 0.03 | 114 | 95.4 |
| storage at 2-8° C. | | | | |
| Post Freeze/thaw | 173 | 0.06 | 113 | 93.9 |

TABLE 2

| | Particle Size Analysis | | mRNA analysis | |
|---|---|---|---|---|
| | Large scale batch 2 | | Large scale batch 2 | |
| | Z- | | mRNA | |
| | Ave | | content | Encapsulation |
| Sample | (nm) | Pdl | (μg/ml) | (%) |
| Post-Mixing | 48.8 | 0.16 | | |
| Post dilution with water (6x) | 47.5 | 0.18 | | |
| Post first ultrafiltration | 51.9 | 0.29 | | |
| Post diafiltration | 65.1 | 0.27 | | |
| Post second ultrafiltration | 74.1 | 0.27 | 1929 | 97.1 |
| Post CF in water | 65.4 | 0.25 | 1942 | 97.4 |
| Post dilution with cryobuffer | 116 | 0.15 | | |
| Post CF in cryobuffer | 129.2 | 0.10 | 146 | 98.2 |
| Post dilution to label claim | 145.6 | 0.10 | 105 | 96.9 |
| Post dilution to label claim (overnight | 151.3 | 0.09 | 111 | 96.9 |
| storage at 2-8° C.) | | | | |
| Post SF | 153.3 | 0.08 | 107 | 96.8 |
| Day 1 filling / Day 1 filling overnight | 154.2 | 0.07 | 103 | 96.5 |
| storage at 2-8° C. | | | | |
| Day 2 filling / Day 2 filling overnight | 167.2 | 0.12 | 106 | 96.4 |
| storage at 2-8° C. | | | | |
| Post Freeze/thaw | 161.1 | 0.13 | 105 | 96.1 |

TABLE 3

| | Particle Size Analysis | | mRNA analysis | |
|---|---|---|---|---|
| | Large scale batch 3 | | Large scale batch 3 | |
| | Z- | | mRNA | |
| | Ave | | content | Encapsulation |
| Sample | (nm) | Pdl | (μg/ml) | (%) |
| Post-Mixing | 59.9 | 0.21 | 75 | 96.8 |
| Post dilution with water (6x) | 58.4 | 0.23 | | |
| Post first ultrafiltration | 68.2 | 0.31 | 659 | 93.6 |
| Post diafiltration | 93.1 | 0.28 | 837 | 91.5 |
| Post second ultrafiltration | 101.6 | 0.28 | 1476 | 93.3 |
| Post CF in water | 91.0 | 0.21 | | |
| Post dilution with cryobuffer | 145.7 | 0.03 | 135 | 90.3 |
| Post CF in cryobuffer | 149.8 | 0.07 | 147 | 95.4 |
| Post dilution to label claim | | | | |
| Post dilution to label claim (overnight | | | | |
| storage at 2-8° C.) | | | | |
| Post SF | 137.2 | 0.09 | | |
| Day 1 filling / Day 1 filling overnight | 146.7 | 0.1 | 135 | 94.8 |
| storage at 2-8° C. | | | | |
| Day 2 filling / Day 2 filling overnight | | | | |
| storage at 2-8° C. | | | | |
| Post Freeze/thaw | 162.4 | 0.05 | 133 | 94.2 |

These tables confirm the reproducibility of the method of the present invention. Moreover, as evidenced from these tables, each of the different steps applied therein contribute to the systematic increase of the mean particle size throughout the method, and allows to achieve large batches of large-sized nanoparticles in the end.

### Example 2—Size-Validation of LNP Batches

In example 1, LNP sizes were determined using dynamic light scattering (DLS). In this example, we validated these particle sizes using a different technology, i.e. Transmission electron microscopy (TEM).

TEM was performed to visualise the ultrastructure and to analyse the size of lipid nanoparticles. The samples evaluated are the ENG batches 1 and 2 (post Freeze/thaw), as defined in tables 1 and 2 of example 1. All samples were kept at −80° C. until the preparation of the grids for the TEM analysis. All experiments were performed at room temperature (20°−25° C.). After thawing the samples, the grids were directly prepared and evaluated in the TEM. Samples were diluted 10× in Tris buffer. Small droplets of the diluted samples were put on 300 mesh carbon-coated copper grids (EM Resolutions, Sheffield, UK) immediately after glow discharging the grids. The nanoparticles were given contrast using a negative stain with 2% uranyl acetate. Once the grids were completely dry (+/−30 min) the samples were visualised and evaluated with a FEI Tecnai Spirit G2 BioTwin TEM (ThermoFisher Scientific, Zaventem, Belgium) at 120 kV. At least 15 images were taken randomly over the entire grid.

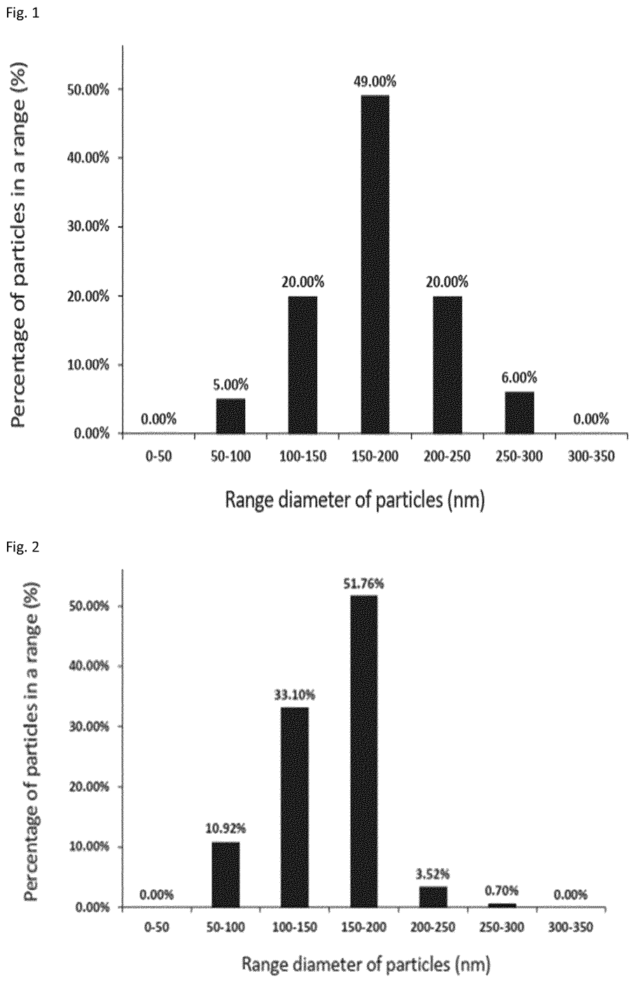

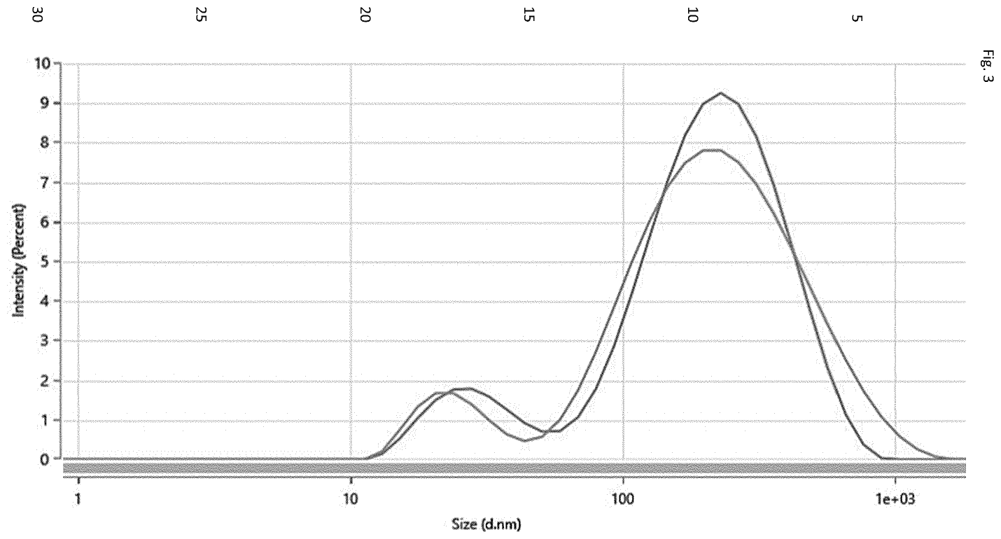

Evaluation of the particle size was performed using FIJI open-source free image analysis software. Segmentation of particles was based on an intensity threshold and the area of each selected particle was measured. Based on this area, the mean diameter for each particle was calculated. The frequency distribution of the diameters of the particles were plotted in excel. Using intervals of 50 nm the diameters between 50 nm and 350 nm were plotted in a graph. The graphs show for each diameter range the percentage of particles belonging to that specific diameter range. FIGS. 1-2 define the size distribution derived from TEM analysis for the LNPs of tables 1-2 of example 1 respectively. As evident from these figures, the particles sizes as determined using DLS (example 1) are very similar to those determined by TEM, thereby confirming the validity of the DLS size determination method.

### Example 3—Effect of Varying the Conditions of the Method of the Invention

One of the main difficulties in the production of large-sized LNPs is their intrinsic instability which is even increased in the event that low amounts of PEG lipids are used, such as intended in the present invention. Therefore, standard methods of the production of small-sized LNPs are not readily transferable to large-sized LNPs, and various parameters of the process needed to be fine-tuned as evidenced by the below experimental details.

Specifically, the inventors found that particular shear rates of above 4000/s were unsuitable during the production process as resulting in precipitation of the product (see table 4).

TABLE 4
effect of varying shear rates during the TFF process

| | TFF process detail | Size* | PDI* | % EE* | Comment |
|---|---|---|---|---|---|
| 1 | Shear rate of 4000 s⁻¹-6000 s⁻¹ TMP < 10 psi. | 152 | 0.10 | 89.6 | Precipitation |
| | Buffer exchange: 4-volumes of cryoprotectant | | | | observed |
| | buffe to wash LNPs using continuous | | | | |
| | diafiltration. Shear rate of 4000 s⁻¹-6000 s⁻¹ and | | | | |
| | TMP < 10 psi. | | | | |
| 2 | Concentration step: Shear rate of 4000 s⁻¹-6000 | 137 | 0.04 | 94.7 | Precipitation |
| | s⁻¹ and TMP < 10 psi. | | | | observed |
| | Buffer exchange: 4-volumes of TBS (~20 mL) to | | | | |
| | wash LNPs using continuous diafiltration. Shear | | | | |
| | rate of 3000 s⁻¹ and TMP < 5 psi. | | | | |
| | Buffer exchange: 4-volumes of cryoprotectant- | | | | |
| | TBS, PH 7.4 (~20 mL) to wash LNPs using | | | | |
| | continuous diafiltration. Shear rate of 3000 s⁻¹ | | | | |
| | and TMP < 5 psi. | | | | |
| 4 | TFF filter: 300 kDa MWCO, 20 cm² | 138 | 0.09 | 76.4 | Precipitation |
| | Concentration step: Shear rate of 4000 s⁻¹- | | | | observed |
| | 6000 s⁻¹ and TMP < 10 psi. | | | | |
| | Buffer exchange: 4-volumes of TBS (20 mL) to | | | | |
| | wash LNPs using continuous diafiltration. Shear | | | | |
| | rate of 3000 s⁻¹ and TMP < 5 psi. | | | | |
| | Buffer exchange: 2-volumes of cryoprotectant- | | | | |
| | TBS, pH 7.4 (~10 mL) to wash LNPs using | | | | |
| | continuous diafiltration. | | | | |
| | Shear rate of 2200 s⁻¹ and TMP < 5 psi. | | | | |
| | Dilute 2X with cryoprotectant-TBS, pH 7.4. | | | | |

*Values based on the end of TFF process.

Two conclusions can be drawn from the data in table 4:
Shear rates (4000/s-6000/s) used for other LNP products are not suitable for the production of large-sized LNPs
Adding TBS and/or TBS with cryoprotectant during the TFF process even with low shear rates leads to precipitation.

Accordingly, these data evidence that in contrast to the claimed shear rate of less than 4000/s, higher shear rates are not suitable in the production of large-sized LNPs.

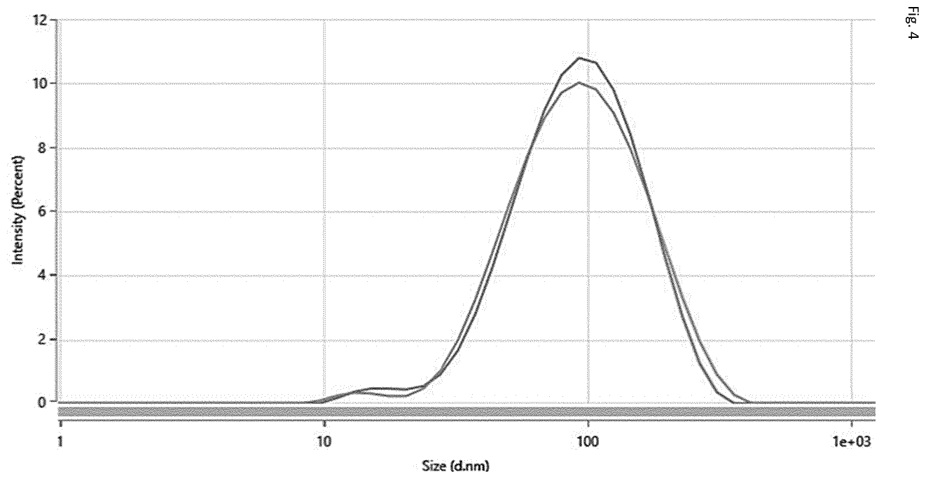

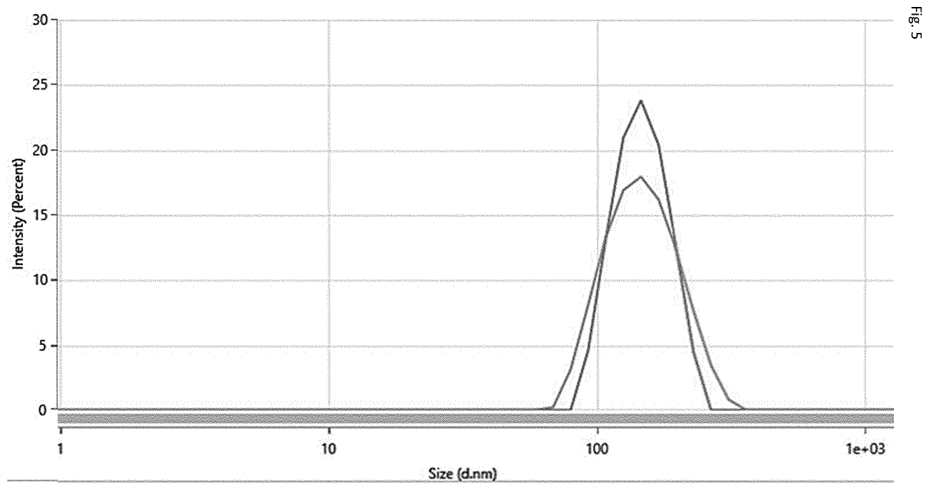

In addition, the inventors found that the shear rate alone proved not to be sufficient for the successful production of the intended LNPs. Specifically, further analysis revealed that 2 different LNP populations were present in the LNP size distribution after the TFF step, and that the PDI value was high (see FIG. 3). Accordingly, the inventors were required to take further measures to obtain the population having the intended LNP particle size. Therefore, the next step in the applied method was a filtration in water (<pH 6.5). However, this filtration step itself significantly reduced the average LNP particle size to below 100 nm as evident from the table 5 and FIG. 4. It was only after further processing steps including dilution in cryobuffer and sterile filtration, that the average particle size of the LNPs again reached at least 140 nm, now with a very low PDI, and a single size population (see table 5 and FIG. 5).

TABLE 5
LNP sizes at different stages during the LNP production process

| | Average-Size | |
|---|---|---|
| After process step | (d · nm) | PDI |
| Post mixing step | 102.9 | 0.35 |
| Bulk diluted in water (6X) | 101.9 | 0.26 |
| TFF in water (shear rates described in | 141 | 0.49 |
| the invention (<4000/s) | | |
| Filtration in water (0.2 μm) | 92.5 | 0.45 |
| Dilution with cryobuffer (10x) - 0.2 μm | 122 | 0.06 |
| After sterile filtration (0.2 μm) | 142 | 0.05 |

In conclusion, both the lower shear rates during TFF and the additional filtration step in water are crucial to obtain the desired quality attributes of the large-sized low-PEG containing LNPs. The combination of TFF in water at low shear rates followed by filtration in water is the only way to get a consistent and robust large scale manufacturing process.

### REFERENCES

Reichmuth et al., 2016—mRNA vaccine delivery using lipid nanoparticles-Therapeutic Delivery Vol. 7, N° 5.
Li et al., 2015—An Orthogonal Array Optimization of Lipid-like Nanoparticles 5 for mRNA Delivery in Vivo-Nano Letters 15 (12) pg. 8099-8107.
Thess et al., 2015—Sequence-engineered mRNA without Chemical Nucleoside Modifications Enables an Effective Protein Therapy in Large Animals—Molecules Therapy Vol. 23, Issue 9 pg. 1456-1464.
Kauffman et al., 2016—Materials for non-viral intracellular delivery of messenger RNA therapeutics—Journal of Controlled Release, Vol. 240, pg. 227-234.
MacLachlan, I. Liposomal formulations for nucleic acid delivery. Antisense Drug Technol. Princ. Strat. Appl. 2007, 2, 237-270.
Richner et al., 2017—Modified mRNA vaccines protect against Zika virus infection—Cell, Vol. 168, Issue 6 pg. 1114-1125
Liang et al., 2017—Efficient Targeting and Activation of Antigen-Presenting Cells In Vivo after 15 Modified mRNA Vaccine Administration in Rhesus Macaques—Molecular Therapy, vol. 25 Issue 12 pg. 2635-2647.
Evers et al., 2018; State-of-the-Art Design and Rapid-Mixing Production Techniques of Lipid Nanoparticles for Nucleic Acid Delivery. Small Methods 2018, 2, 1700375
Chen, S., Tam, Y. Y. C., Lin, P. J., Sung, M. M., Tam, Y. K. and Cullis, P. R., 2016. Influence of particle size on the in vivo potency of lipid nanoparticle formulations of siRNA. Journal of Controlled Release, 235, pp. 236-244.

1. A method for the large-scale production of a composition comprising lipid nanoparticles (LNPs) having an average diameter of at least 140 nm, said method comprising the steps of:
a) mixing a first composition comprising an ionisable lipid, a phospholipid, a sterol, and a PEG lipid in an organic solvent with a second composition comprising one or more active agents in an acidic buffer, thereby obtaining a post-mixing bulk,
b) applying tangential flow filtration (TFF) to said post-mixing bulk of steps a),
c) applying a filtration step in an aqueous solution having pH<6.5 to said composition of step b),
d) adding a cryobuffer comprising a cryoprotectant to said composition of step c),
e) applying a filtration step in an aqueous solution having a pH>6.5 to said composition of step d),
f) sterile filtration of said composition of step e), thereby obtaining a composition of lipid nanoparticles having an average diameter of at least 140 nm and containing said active agent;
characterized in that said TFF of step b) is performed at a shear rate of less than 4000/s, and wherein LNPs comprise less than 1 mol % of PEGylated lipid.

2. The method of claim 1 wherein in step a), the mixing flow rate of the first composition is lower than the mixing flow rate of the second composition.

3. The method of claim 1 or 2 wherein in step a), the ratio of the mixing flow rates between the first and the second composition is about 1:2.

4. The method of any of the claims 1-3 wherein the tangential flow filtration in step b) comprises a first ultrafiltration step being performed at a lower shear rate compared to the other tangential flow filtration step.

5. The methods of any of the claims 1-4 wherein the tangential flow filtration in step b) comprises a first ultrafiltration step being performed at a shear rate of <3000/s whilst the other tangential flow filtration steps are performed at a shear rate of <4000/s.

6. The method of any of the claims 1-5 wherein the organic solvent is chloroform or an alcohol such as methanol or ethanol; in particular ethanol.

7. The method of any of the claims 1-6 wherein said active agent is selected from the list comprising: small molecules and biomolecules such as nucleic acids, antibodies, proteins, peptides; in particular mRNA molecules.

8. The method of any of the claims 1-7 wherein the cryoprotectant is selected from the list comprising maltose, maltodextrin, dextran, mannitol, trehalose, glucose or sucrose; in particular trehalose or sucrose.

9. The method of any of the claims 1-8 wherein the cryobuffer comprises a cryoprotectant and one or more of citrate, Tris and PBS; in particular one or more TRIS or PBS.

10. The method of any of the claims 1-9 wherein the first composition of step a) is at a temperature of 30-45° C.

11. The method of any of the claims 1-10 wherein the post-mixing bulk obtained in step a) is diluted before step b).

12. The method of any of the claims 1-11 wherein the composition obtained in step e) is diluted before step f).

13. The method of any of the claims 1-12 wherein the filter size of the filtration in steps c and/or e) is 0.20 μm.

14. The method of any of the claims 1-13, wherein said aqueous solution of steps c) and/or e) is water, in particular water for injection.

15. The method of any of the claims 1-14, wherein said filtration of steps c) and/or e) is a clarifying filtration.
